(19) United States
(12) Patent Application Publication
(10) Pub. No.: US 2012/0027743 A1
(43) Pub. Date: Feb. 2, 2012

(54) Method for the Treatment of Hemophilia

(75) Inventors: Haiyan Jiang, Belmont, MA (US); Tongyao Liu, Alameda, CA (US); Xin Zhang, San Diego, CA (US)

(73) Assignee: BAYER HEALTHCARE LLC, Tarrytown, NY (US)

(21) Appl. No.: 13/127,461

(22) PCT Filed: Nov. 3, 2009
(86) PCT No.: PCT/US2009/063151

§ 371 Date: Oct. 17, 2011

Related U.S. Application Data
(60) Provisional application No. 61/110,809, filed on Nov. 3, 2008.

(51) Int. Cl. A61K 38/48; A61P 7/04; A61K 38/37

(52) U.S. Cl. 424/94.3; 514/14.1

(57) ABSTRACT

The present invention is directed to a method for the treatment of hemophilia.

### METHOD FOR THE TREATMENT OF HEMOPHILIA

This application claims benefit of U.S. Provisional Application Ser. No. 61/110,809; filed on Nov. 3, 2008, the contents of which are incorporated herein by reference in their entirety.

### FIELD OF THE INVENTION

The present invention is directed to a method for the treatment of hemophilia.

### BACKGROUND OF THE INVENTION

Hemophilia A is the most common hereditary coagulation disorder with an estimated incidence of 1 per 5,000 males. It is caused by a deficiency or structural defects in Factor VIII (FVIII), a component of the intrinsic pathway of blood coagulation. Human FVIII has been produced recombinantly, and has been shown to be effective as a replacement therapy for hemophilia A.

The current treatment for hemophilia A involves intravenous injection or infusion of FVIII. Patients may be treated either when a bleeding episode occurs (“on-demand therapy”) or as a prophylactic therapy administered several times a week. For example, FVIII may be given three times per week for prophylactic treatment. In addition, venous access devices may be surgically implanted for administration. However, infection can be a problem for these devices. As such, these cumbersome modes of administration create tremendous barriers for patient compliance.

Therefore, there is a need to develop alternative modes of administration to encourage patient compliance. The present invention provides such method of treatment.

### SUMMARY OF THE INVENTION

The present invention is directed to a method of treating hemophilia comprising subcutaneously administering an effective amount of a coagulation factor or mutein thereof covalently attached at one or more amino acid sites to one or more biocompatible polymers. Examples of coagulation factors include, but are not limited to, FVIII, Factor VII (FVII), or Factor IX (FIX). The one or more biocompatible polymers may be attached at random sites or may be site-specific.

In one embodiment, the biocompatible polymer is selected from polyalkylene oxides, dextrans, colominic acids, carbohydrate-based polymers, polymers of amino acids, biotin derivatives, polyvinyl alcohol, polycarboxylates, polyvinylpyrrolidone, polyethylene-co-maleic acid anhydride, polystyrene-co-malic acid anhydride, polyoxazoline, polyacryloylmorpholine, heparin, albumin, celluloses, hydrolysates of chitosan, starches, glycogen, agaroses and derivatives thereof, guar gum, pullulan, inulin, xanthan gum, carrageenan, pectin, and alginic acid hydrolysates. As an example, the polyalkylene oxide may be polyethylene glycol. Furthermore, the polyethylene glycol may have a size range from 5 kDa to 150 kDa or greater.

In another embodiment, the biocompatible polymer is a starch such as hydroxyethyl starch or hydroxypropyl starch. As an example, the size range for hydroxyethyl starch may be 150 kDa or greater.

In a further embodiment, the biocompatible polymer is covalently attached at a predefined site on the coagulation factor or mutein thereof. For example, the biocompatible polymer may be covalently attached to one or more amino acid sites of the FVIII polypeptide or FVIII mutein selected from 81, 129, 377, 378, 468, 487, 491, 504, 556, 570, 711, 1648, 1795, 1796, 1803, 1804, 1808, 1810, 1864, 1903, 1911, 2091, 2118 and 2284.

In one embodiment, the FVIII mutein is B-domain deleted factor VIII. In another embodiment, the FVIII mutein further comprises one or more amino acid substitutions selected from 81, 129, 377, 378, 468, 487, 491, 504, 556, 570, 711, 1648, 1795, 1796, 1803, 1804, 1808, 1810, 1864, 1903, 1911, 2091, 2118 and 2284. For example, the amino acid substitution is cysteine.

In another embodiment, the biocompatible polymer is covalently attached at a random or predefined site on FVII or FIX, or a mutein thereof.

In another embodiment, the coagulation factor or mutein is administered prophylactically. In a further embodiment, the coagulation factor or mutein is administered daily with a initial loading dose followed by low maintenance doses. In another embodiment, the coagulation factor or mutein is administered in a dose to sustain a trough levels of approximately 1-2% of normal

### DESCRIPTION OF THE FIGURES

FIG. 1. Factor VIII was intradermally administered to naive HemA mice. Plasma FVIII activities were then determined by Coatest assay.

### DESCRIPTION OF THE INVENTION

It is to be understood that this invention is not limited to the particular methodology, protocols, cell lines, animal species or genera, constructs, and reagents described and as such may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a,” “and,” and “the” include plural reference unless the context clearly dictates otherwise. Thus, for example, reference to “an agent” is a reference to one or more agents and includes equivalents thereof known to those skilled in the art, and so forth.

Unless defined otherwise, all technical and scientific terms used herein have the same meaning as commonly understood to one of ordinary skill in the art to which this invention belongs. Although any methods, devices, and materials similar or equivalent to those described herein can be used in the practice or testing of the invention, the preferred methods, devices and materials are now described.

All publications and patents mentioned herein are hereby incorporated herein by reference for the purpose of describing and disclosing, for example, the methodologies that are described in the publications which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

Prophylactic treatment for hemophilia A requires frequent intravenous injections or infusions of FVIII necessitated by its short half-life (8-12 hrs) in vivo. Frequent intravenous injections with large volumes are inconvenient, difficult to administer to young children, and often result in venous catheter-related infection.

Subcutaneous administration affords an alternative mode of administration and provides an unmet medical need. However, subcutaneous injectable FVIII as a treatment in humans is currently not feasible, largely due to its extremely low bioavailability (<5%). Low bioavailability requires high doses which is economically prohibitive. Furthermore, limitations in injection volume necessitate a highly concentrated formulation which is technically challenging.

As described herein, PEGylation of FVIII significantly improved the bioavailability of intradermally administered FVIII in a hemophilia A mouse model. Intradermal administration in mice is analogous to subcutaneous injection in humans.

The present invention demonstrates that FVIII or a mutein thereof covalently attached at one or more amino acid sites to one or more biocompatible polymers achieves improved recovery of functionally active FVIII in vivo.

The present invention is directed to a coagulation factor or mutein thereof conjugated to one or more biocompatible polymers such as polyethylene glycol (PEG), hydroxyethyl starch (HES), polysialic acid (PSA), other hydrophilic polymers, or FVIII formulated with hydrophilic polymer. These conjugates may be administered subcutaneously for the prophylactic treatment of hemophilia A. In addition, the conjugates may be subcutaneously administered weekly or may be administered daily with an initial loading dose followed by small volume of low maintenance doses to sustain a steady efficacious trough levels of approximately 1-2% of normal.

A biocompatible polymer includes, but is not limited to, polyalkylene oxides such as without limitation polyethylene glycol (PEG), methoxypolyethylene glycol (mPEG), dextrans, colominic acids or other carbohydrate based polymers, polymers of amino acids, biotin derivatives, polyvinyl alcohol (PVA), polycarboxylates, polyvinylpyrrolidone, polyethylene-co-maleic acid anhydride, polystyrene-co-malic acid anhydride, polyoxazoline, polyacryloylmorpholine, heparin, albumin, celluloses, hydrolysates of chitosan, starches such as hydroxyethyl-starches and hydroxy propyl-starches, glycogen, agaroses and derivatives thereof, guar gum, pullulan, inulin, xanthan gum, carrageenan, pectin, alginic acid hydrolysates, other bio-polymers and any equivalents thereof. Other useful polyalkylene glycol compounds are polypropylene glycols (PPG) , polybutylene glycols (PBG), PEG-glycidyl ethers (Epox-PEG), PEG-oxycarbonylimidazole (CDI-PEG), branched polyethylene glycols, linear polyethylene glycols, forked polyethylene glycols and multi-armed or “super branched” polyethylene glycols (star-PEG).

“PEG” and “polyethylene glycol” as used herein are interchangeable and include any water-soluble poly(ethylene oxide). Typically, PEGs for use in accordance with the invention comprise the following structure “—(OCH₂CH₂) ₙO—” where (n) is 2 to 4000. As used herein, PEG also includes “—CH₂CH₂—O(CH₂CH₂O)ₙ—CH₂CH₂—” and “—(OCH₂CH₂)ₙO—,” depending upon whether or not the terminal oxygens have been displaced. Throughout the specification and claims, it should be remembered that the term “PEG” includes structures having various terminal or “end capping” groups, such as without limitation a hydroxyl or a C₁₋₂₀ alkoxy group. The term “PEG” also means a polymer that contains a majority, that is to say, greater than 50%, of —OCH₂CH₂— repeating subunits. With respect to specific forms, the PEG can take any number of a variety of molecular weights, as well as structures or geometries such as branched, linear, forked, and multifunctional.

PEGylation is the covalent attachment of long-chained polyethylene glycol (PEG) molecules to a protein or other molecule. The PEG may be in a linear form or in branched form. In addition, PEGylation may be random (e.g., targeting primary amines such as N-terminus and lysines) or site-specific (e.g., targeting specific amino acids).

For example, in one embodiment of the invention, the biocompatible polymer (e.g., PEG) is covalently attached to the FVIII at one or more of amino acid positions such as, but not limited to, 81, 129, 377, 378, 468, 487, 491, 504, 556, 570, 711, 1648, 1795, 1796, 1803, 1804, 1808, 1810, 1864, 1903, 1911, 2091, 2118 and 2284.

Examples of coagulation factors include, but are not limited to, FVII, FVIII, FIX, and muteins thereof.

Factor VIII includes the human full-length FVIII molecule. Muteins of FVIII include, for example, but not limited to, B domain deleted FVIII (BDD), functionally active FVIII fragments, and a FVIII molecule or fragment thereof comprising one or more amino acid substitutions at positions 81, 129, 377, 378, 468, 487, 491, 504, 556, 570, 711, 1648, 1795, 1796, 1803, 1804, 1808, 1810, 1864, 1903, 1911, 2091, 2118 and 2284. As an example, the amino acid substitution may include cysteine at one or more positions 81, 129, 377, 378, 468, 487, 491, 504, 556, 570, 711, 1648, 1795, 1796, 1803, 1804, 1808, 1810, 1864, 1903, 1911, 2091, 2118 and 2284.

Examples of additional FVIII muteins and methods of producing such muteins are described in U.S. Patent Application Publication No. 2006/0115876, which is incorporated herein.

Examples of FVII include the human full-length FVII molecule as well as muteins of FVII described in WO 99/20767, WO 00/66753, WO 01/58935, WO 03/093465, WO 04/029091, WO 04/083361, and WO 04/111242.

Examples of FIX include the human full-length FIX molecule as well as muteins of FIX described in U.S. Pat. No. 6,531,298; U.S. patent application Ser. No. PCT/US09/40691; and U.S. patent application Ser. No. PCT/US09/40813.

Production of Muteins

A mutein is a genetically engineered protein arising as a result of a laboratory induced mutation to a protein or polypeptide.

Amino acid sequence alteration may be accomplished by a variety of techniques such as, for example, by modifying the corresponding nucleic acid sequence by site-specific mutagenesis. Techniques for site-specific mutagenesis are well known in the art and are described in, for example, Zoller, et al., (DNA 3:479-488, 1984) or Horton, et al., (Gene 77:61-68, 1989, pp. 61-68). For example, a conservative substitution is recognized in the art as a substitution of one amino acid for another amino acid that has similar properties and include, for example, the changes of alanine to serine or arginine to lysine. Thus, using the nucleotide and amino acid sequences of FVIII, FVII, or FIX one may introduce the alteration(s) of choice. Likewise, procedures for preparing a DNA construct using polymerase chain reaction using specific primers are well known to persons skilled in the art (see, e.g., PCR Protocols, 1990, Academic Press, San Diego, Calif., USA).

The nucleic acid construct encoding the muteins may also be prepared synthetically by established standard methods, for example, the phosphoramidite method described by Beaucage, et al., (Gene Amplif. Anal. 3:1-26, 1983). According to the phosphoamidite method, oligonucleotides are synthesized, for example, in an automatic DNA synthesizer, purified, annealed, ligated, and cloned in suitable vectors. The DNA sequences encoding the muteins may also be prepared by polymerase chain reaction using specific primers, for example, as described in U.S. Pat. No. 4,683,202, or Saiki, et al., (Science 239:487-491, 1988). Furthermore, the nucleic acid construct may be of mixed synthetic and genomic, mixed synthetic and cDNA, or mixed genomic and cDNA origin prepared by ligating fragments of synthetic, genomic, or cDNA origin (as appropriate), corresponding to various parts of the entire nucleic acid construct, in accordance with standard techniques.

The DNA sequences encoding the muteins may be inserted into a recombinant vector using recombinant DNA procedures. The choice of vector will often depend on the host cell into which the vector is to be introduced. The vector may be an autonomously replicating vector or an integrating vector. An autonomously replicating vector exists as an extrachromosomal entity and its replication is independent of chromosomal replication, for example, a plasmid. An integrating vector is a vector that integrates into the host cell genome and replicates together with the chromosome(s) into which it has been integrated.

The vector may be an expression vector in which the DNA sequence encoding the mutein is operably linked to additional segments required for transcription, translation, or processing of the DNA, such as promoters, terminators, and polyadenylation sites. In general, the expression vector may be derived from plasmid or viral DNA, or may contain elements of both. The term “operably linked” indicates that the segments are arranged so that they function in concert for their intended purposes, for example, transcription initiates in a promoter and proceeds through the DNA sequence coding for the polypeptide.

Expression vectors for use in expressing the muteins may comprise a promoter capable of directing the transcription of a cloned gene or cDNA. The promoter may be any DNA sequence that shows transcriptional activity in the host cell of choice and may be derived from genes encoding proteins either homologous or heterologous to the host cell. Examples of suitable promoters for directing the transcription of the DNA encoding the mutein in mammalian cells are, for example, the SV40 promoter (Subramani, et al., Mol. Cell Biol. 1:854-864, 1981), the MT-I (metallothionein gene) promoter (Palmiter, et al., Science 222:809-814, 1983), the CMV promoter (Boshart, et al., Cell 41:521-530, 1985), the myeloproliferative sarcoma virus (MPSV) LTR promoter (Lin, et al., Gene. 147:287-92, 1994), or the adenovirus 2 major late promoter (Kaufman, et al., Mol. Cell. Biol. 2:1304-1319, 1982).

The DNA sequences encoding the mutein may also, if necessary, be operably connected to a suitable terminator, such as the human growth hormone terminator (Palmiter, et al., Science 222:809-814, 1983) or TPII (Alber, et al., J. Mol. Appl. Gen. 1:419-434, 1982), or ADH3 (McKnight, et al., EMBO J. 4:2093-2099, 1985) terminators. The expression vectors may also contain a polyadenylation signal located downstream of the insertion site. Polyadenylation signals include the early or late polyadenylation signal from SV40, the polyadenylation signal from the adenovirus 5 Elb region, the human growth hormone gene terminator (DeNoto, et al., Nucl. Acids Res. 9:3719-3730, 1981), or the polyadenylation signal from the human TF gene or the human thrombomodulin gene. The expression vectors may also include enhancer sequences, such as the SV40 enhancer.

The procedures used to ligate the DNA sequences coding for the muteins, the promoter, and optionally the terminator and to insert them into suitable vectors containing the information necessary for replication, are well known to persons skilled in the art (see, e.g., Sambrook, et al., Molecular Cloning: A Laboratory Manual, Cold Spring Harbor, N.Y., 1989).

Methods of transfecting mammalian cells and expressing DNA sequences introduced into the cells are described in, for example, Kaufman, et al., (J. Mol. Biol. 159:601-621, 1982); Southern, et al., (J. Mol. Appl. Genet. 1:327-341, 1982); Loyter, et al., (Proc. Natl. Acad. Sci. USA 79:422-426, 1982); Wigler, et al., (Cell 14:725-731, 1978); Corsaro, et al., (Somatic Cell Genetics 7:603-616, 1981), Graham, et al., (Virology 52:456-467, 1973); and Neumann, et al., (EMBO J. 1:841-845, 1982). Cloned DNA sequences may be introduced into cultured mammalian cells by, for example, lipofection, DEAE-dextran-mediated transfection, microinjection, protoplast fusion, calcium phosphate precipitation, retroviral delivery, electroporation, sonoporation, laser irradiation, magnetofection, natural transformation, and biolistic transformation (see, e.g., Mehier-Humbert, et al., Adv. Drug Deliv. Rev. 57:733-753, 2005). To identify and select cells that express the exogenous DNA, a gene that confers a selectable phenotype (a selectable marker) is generally introduced into cells along with the gene or cDNA of interest. Selectable markers include, for example, genes that confer resistance to drugs such as neomycin, puromycin, hygromycin, and methotrexate. The selectable marker may be an amplifiable selectable marker, which permits the amplification of the marker and the exogenous DNA when the sequences are linked. Exemplary amplifiable selectable markers include dihydrofolate reductase (DHFR) and adenosine deaminase. It is within the purview of one skilled in the art to choose suitable selectable markers (see, e.g., U.S. Pat. No. 5,238,820).

After cells have been transfected with DNA, they are grown in an appropriate growth medium to express the gene of interest. As used herein the term “appropriate growth medium” means a medium containing nutrients and other components required for the growth of cells and the expression of the mutein.

Media generally include, for example, a carbon source, a nitrogen source, essential amino acids, essential sugars, vitamins, salts, phospholipids, protein; and growth factors may also be provided. Drug selection is then applied to select for the growth of cells that express the selectable marker in a stable fashion. For cells that have been transfected with an amplifiable selectable marker, the drug concentration may be increased to select for an increased copy number of the cloned sequences, thereby increasing expression levels. Clones of stably transfected cells are then screened for expression of the mutein.

Examples of mammalian cell lines for use in the present invention are the COS-1 (ATCC CRL 1650), baby hamster kidney (BHK), HKB11 (Cho, et al., J. Biomed. Sci, 9:631-638, 2002), and HEK-293 (ATCC CRL 1573; Graham, et al., J. Gen. Virol. 36:59-72, 1977) cell lines. In addition, a number of other cell lines may be used within the present invention, including rat Hep I (rat hepatoma; ATCC CRL 1600), rat Hep II (rat hepatoma; ATCC CRL 1548), TCMK-1 (ATCC CCL 139), Hep-G2 (ATCC HB 8065), NCTC 1469 (ATCC CCL 9.1), CHO-K1 (ATCC CCL 61), and CHO-DUKX cells (Urlaub, et al., Proc. Natl. Acad. Sci. USA 77:4216-4220, 1980).

The muteins may be recovered from cell culture medium and may then be purified by a variety of procedures known in the art including, but not limited to, chromatography (e.g., ion exchange, affinity, hydrophobic, chromatofocusing, and size exclusion), electrophoretic procedures (e.g., preparative isoelectric focusing (IEF), differential solubility (e.g., ammonium sulfate precipitation)), extraction (see, e.g., Protein Purification, Janson and Lars Ryden, editors, VCH Publishers, New York, 1989), or various combinations thereof. Additional purification may be achieved by conventional chemical purification means, such as high performance liquid chromatography. Other methods of purification are known in the art, and may be applied to the purification of the muteins (see, e.g., Scopes, R., Protein Purification, Springer-Verlag, N.Y., 1982).

Generally, “purified” shall refer to a protein, polypeptide, or peptide composition that has been subjected to fractionation to remove various other components, and which substantially retains its expressed biological activity. Where the term “substantially purified” is used, this designation shall refer to a composition in which the protein, polypeptide, or peptide forms the major component of the composition, such as constituting about 50%, about 60%, about 70%, about 80%, about 90%, about 95%, about 99%, or more of the proteins in the composition.

Various methods for quantifying the degree of purification of a protein are known to those of skill in the art. These include, for example, determining the specific activity of an active fraction, or assessing the amount of polypeptides within a fraction by SDS/PAGE analysis. An exemplary method for assessing the purity of a fraction is to calculate the specific activity of the fraction, compare the activity to the specific activity of the initial extract, and to thus calculate the degree of purity, herein assessed by a “-fold purification number.” The actual units used to represent the amount of activity will, of course, be dependent upon the particular assay technique.

“Homology” refers to the degree of similarity between two protein or polynucleotide sequences. The correspondence between two sequences may be determined by techniques known in the art. For example, homology may be determined by a direct comparison of the sequence information of the polynucleotide or protein sequences. Usually, two sequences may be homologous if the sequences exhibit at least 75% sequence identity, 80% sequence identity, 85% sequence identity, 90% sequence identity, or 95% sequence identity.

To determine the percent homology of two protein sequences, or of two polynucleotide sequences, the sequences are aligned for optimal comparison purposes. For example, gaps may be introduced in the sequence of one protein or polynucleotide for optimal alignment with the other protein or polynucleotide. The amino acid residues or nucleotides at corresponding amino acid positions or nucleotide positions are then compared. When a position in one sequence is occupied by the same amino acid residue or nucleotide as the corresponding position in the other sequence, then the molecules are homologous at that position. As used herein, amino acid or nucleic acid “homology” is equivalent to amino acid or nucleic acid “identity.” The percent homology between the two sequences is a function of the number of identical positions shared by the sequences, that is, the percent homology equals the number of identical positions/total number of positions times 100.

The invention also encompasses muteins having a lower degree of identity, but having sufficient similarity so as to perform one or more of the same functions performed by the muteins of the invention. Similarity is determined by conserved amino acid substitution. Such substitutions are those that substitute a given amino acid in a protein by another amino acid of like characteristics. Typically seen as conservative substitutions are the replacements, one for another, among the aliphatic amino acids Ala, Val, Leu, and Ile; interchange of the hydroxyl residues Ser and Thr; exchange of the acidic residues Asp and Glu; substitution between the amide residues Asn and Gln; exchange of the basic residues Lys and Arg and replacements among the aromatic residues Phe, Trp, and Tyr.

The single letter abbreviation for a particular amino acid, its corresponding amino acid, and three letter abbreviation are as follows: A, alanine (Ala); C, cysteine (Cys); D, aspartic acid (Asp); E, glutamic acid (Glu); F, phenylalanine (Phe); G, glycine (Gly); H, histidine (His); I, isoleucine (Ile); K, lysine (Lys); L, leucine (Leu); M, methionine (Met); N, asparagine (Asn); P, proline (Pro); Q, glutamine (Gin); R, arginine (Arg); S, serine (Ser); T, threonine (Thr); V, valine (Val); W, tryptophan (Trp); Y, tyrosine (Tyr); and norleucine (Nle).

Both identity and similarity can be readily calculated (Computational Molecular Biology, Lesk, A. M., ed., Oxford University Press, New York, 1988; Biocomputing: Informatics and Genome Projects, Smith, D. W., ed., Academic Press, New York, 1993; Computer Analysis of sequence Data, Part 1, Griffin, A. M., and Griffin, H. G., eds., Humana Press, New Jersey, 1994; Sequence Analysis in Molecular Biology, von Heinje, G., Academic Press, 1987; and Sequence Analysis Primer, Gribskov, M. and Devereux, J., eds., M. Stockton Press, New York, 1991). Computer program methods to determine identity and similarity between two sequences include, but are not limited to, GCG program package (Devereux, et al., Nucleic Acids Res. 12:387, 1984), BLASTP, BLASTN, FASTA (Atschul, et al., J. Molec. Biol. 215:403, 1990).

A mutein can differ in amino acid sequence by one or more substitutions, deletions, insertions, inversions, fusions, and truncations or a combination of any of these. In addition, a variation may provide a peptide tag or peptide expression tag that is incorporated the mutein. The peptide tag can be a FLAG tag, a c-myc tag, an E-tag, a 6×His tag, or similar peptide tag. The peptide tag may occur at the N-terminus, the C-terminus or elsewhere in the mutein. The peptide tag is useful both in vivo and in vitro for detection, purification, or identification of the mutein. It will be generally understood by one skilled it the art that the peptide tag sequence will usually be removed from the sequence used in the preparation or expression of the final drug substance.

Methods of Use

As used herein, various terms are defined below.

The term “treatment” includes any process, action, application, therapy, or the like, wherein a subject (or patient), including a human being, is provided medical aid with the object of improving the subject's condition, directly or indirectly, or slowing the progression of a condition or disorder in the subject.

The phrase “therapeutically effective” means the amount of agent administered that will achieve the goal of improvement in a disease, condition, and/or disorder severity, while avoiding or minimizing adverse side effects associated with the given therapeutic treatment.

The term “pharmaceutically acceptable” means that the subject item is appropriate for use in a pharmaceutical product.

Accordingly, an embodiment of this invention includes a method of treating hemophilia in a patient which comprises subcutaneous administration to said patient a composition containing an amount of conjugated FVIII, FVII, FIX, or mutein thereof.

The term “combination therapy” or “co-therapy” means the administration of two or more therapeutic agents to treat a disease, condition, and/or disorder. Such administration encompasses co-administration of two or more therapeutic agents in a substantially simultaneous manner or administration of each type of therapeutic agent in a sequential manner.

Combination therapy includes administration of a single pharmaceutical dosage formulation which contains conjugated FVIII, FVII, FIX, or mutein thereof and one or more additional therapeutic agents, as well as administration of conjugated FVIII, FVII, FIX, or mutein thereof and each additional therapeutic agents in its own separate pharmaceutical dosage formulation. For example, conjugated FVIII, FVII, FIX, or mutein thereof and a therapeutic agent may be administered to the patient together in a single dosage composition or each agent may be administered in separate dosage formulations.

Where separate dosage formulations are used, the conjugated FVIII, FVII, FIX, or mutein thereof and one or more additional therapeutic agents may be administered at essentially the same time (e.g., concurrently) or at separately staggered times (e.g., sequentially).

Pharmaceutical Compositions

Conjugated FVIII, FVII, FIX, or mutein thereof as described herein may be provided in a pharmaceutical composition comprising a pharmaceutically acceptable carrier. The pharmaceutically acceptable carrier may be non-pyrogenic. The compositions may be administered alone or in combination with at least one other agent, such as stabilizing compound, which may be administered in any sterile, biocompatible pharmaceutical carrier including, but not limited to, saline, buffered saline, dextrose, and water. A variety of aqueous carriers may be employed including, but not limited to saline, glycine, or the like. These solutions are sterile and generally free of particulate matter. These solutions may be sterilized by conventional, well known sterilization techniques (e.g., filtration). The compositions may contain pharmaceutically acceptable auxiliary substances as required to approximate physiological conditions such as pH adjusting and buffering agents, and the like. The concentration of conjugated FVIII, FVII, FIX, or mutein thereof in such pharmaceutical formulation may vary widely, and may be selected primarily based on fluid volumes, viscosities, etc., according to the particular mode of administration.

The compositions may be administered to a patient alone, or in combination with other agents, drugs or hormones. In addition to the active ingredients, these pharmaceutical compositions may contain suitable pharmaceutically acceptable carriers comprising excipients and auxiliaries that facilitate processing of the active compounds into preparations which may be used pharmaceutically. Pharmaceutical compositions of the invention may be administered by subcutaneous means.

Formulations suitable for subcutaneous, intravenous, intramuscular, and the like; suitable pharmaceutical carriers; and techniques for formulation and administration may be prepared by any of the methods well known in the art (see, e.g., Remington's Pharmaceutical Sciences, Mack Publishing Co., Easton, Pa., 20^{th} edition, 2000).

Determination of a Therapeutically Effective Dose

The determination of a therapeutically effective dose is well within the capability of those skilled in the art. A therapeutically effective dose refers to the amount of an agent that may be used to effectively treat a disease (e.g., hemophilia) compared with the efficacy that is evident in the absence of the therapeutically effective dose.

The therapeutically effective dose may be estimated initially in animal models (e.g., rats, mice, rabbits, dogs, or pigs). The animal model may also be used to determine the appropriate concentration range and route of administration. Such information may then be used to determine useful doses and routes for administration in humans.

The exact dosage may be determined by the practitioner, in light of factors related to the patient who requires treatment. Dosage and administration may be adjusted to provide sufficient levels of the agent or to maintain the desired effect. Factors that may be taken into account include the severity of the disease state, general health of the subject, age, weight, and gender of the subject, diet, time and frequency of administration, drug combination(s), reaction sensitivities, and tolerance/response to therapy.

All patents and patent applications cited in this disclosure are expressly incorporated herein by reference. The above disclosure generally describes the present invention. A more complete understanding can be obtained by reference to the following specific examples, which are provided for purposes of illustration only and are not intended to limit the scope of the invention.

### EXAMPLES

In order that this invention may be better understood, the following examples are set forth. These examples are for the purpose of illustration only, and are not to be construed as limiting the scope of the invention in any manner. All publications mentioned herein are incorporated by reference in their entirety.

### Example 1

### Analysis of Activity of Intradermally Administered FVIII

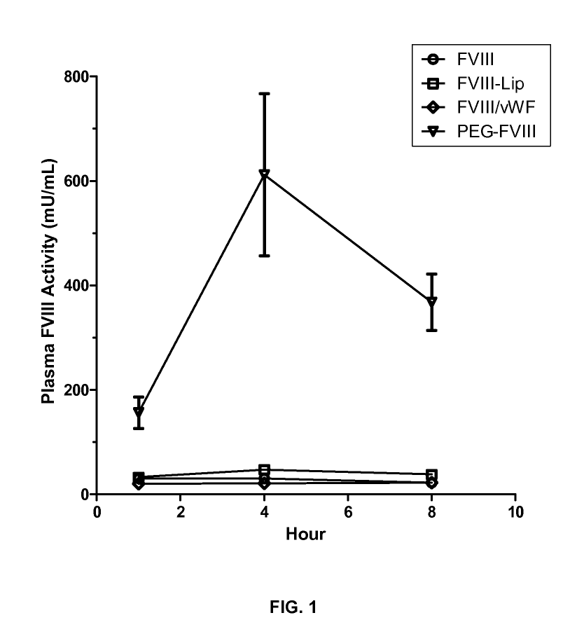

Naive Hemophilia A mice were intradermally administered with 13 IU/mouse of rFVIII, 14 IU/mouse of rFVIII formulated in PEG-Liposome (FVIII-Lip), 12 IU/mouse of PEGylated FVIII (PEG-FVIII), and 15 IU/mouse of rFVIII premixed with vWF at a molar ratio of 1:2. Animals were then euthanized at 1, 4, and 8 hrs post dosing (3 mice per treatment per time point) and blood samples were obtained. Plasma FVIII activities were then determined by Coatest assay. The results are shown in FIG. 1.

In comparison to rFVIII, FVIII-Lip, and FVIII/vWF complex, which have only marginally detectable levels of plasma FVIII activity, and which are approximately 0.1-0.4% of the respective input dose at all three time points examined, PEG-FVIII achieved on average 10-fold higher recovery ranging from 1-5% of the input dose at all time points.

1. A method of treating hemophilia comprising subcutaneously or intradermally administering to a patient in need thereof an effective amount of a coagulation factor or a mutein thereof covalently attached at one or more amino acid sites to one or more biocompatible polymers.

2. The method of claim 1, wherein the biocompatible polymer is selected from polyalkylene oxides, dextrans, colominic acids, carbohydrate-based polymers, polymers of amino acids, biotin derivatives, polyvinyl alcohol, polycarboxylates, polyvinylpyrrolidone, polyethylene-co-maleic acid anhydride, polystyrene-co-malic acid anhydride, polyoxazoline, polyacryloylmorpholine, heparin, albumin, celluloses, hydrolysates of chitosan, starches, glycogen, agaroses and derivatives thereof, guar gum, pullulan, inulin, xanthan gum, carrageenan, pectin, and alginic acid hydrolysates.

3. The method of claim 2, wherein the polyalkylene oxide comprises polyethylene glycol.

4. The method of claim 3, wherein the polyethylene glycol comprises methoxypolyethylene glycol.

5. The method of claim 3, wherein the methoxypolyethylene glycol has a size range from 5 kDa to 150 kDa.

6. The method of claim 2, wherein the starch comprises hydroxyethyl starch and hydroxypropyl starch.

7. The method of claim 1, wherein the biocompatible polymer is covalently attached at a predefined site on the coagulation factor or mutein thereof.

8. The method of claim 1, wherein said coagulation factor is selected from FVII, FVIII, and FIX, and muteins thereof.

9. The method of claim 8, wherein the biocompatible polymer is covalently attached to FVIII at one or more amino acid sites selected from 81, 129, 377, 378, 468, 487, 491, 504, 556, 570, 711 1648, 1795, 1796, 1803, 1804, 1808, 1810, 1864, 1903, 1911, 2091, 2118 and 2284.

10. The method of claim 9, wherein the one or more sites for biocompatible polymer attachment are substituted by site specific cysteine mutation.

11. The method of claim 8, wherein FVIII is B-domain deleted FVIII.

12. The method of claim 11, wherein B-domain deleted FVIII comprises one or more amino acid substitutions selected from 81, 129, 377, 378, 468, 487, 491, 504, 556, 570, 711, 1648, 1795, 1796, 1803, 1804, 1808, 1810, 1864, 1903, 1911, 2091, 2118 and 2284.

13. The method of claim 12, wherein a biocompatible polymer is covalently attached to B-domain deleted FVIII at one or more amino acid site substitutions.

14. The method of claim 1, wherein coagulation factor or mutein thereof is administered prophylactically.
